(19) United States
(12) Patent Application Publication
(10) Pub. No.: US 2010/0130554 A1
(43) Pub. Date: May 27, 2010

(54) USE OF 4-CYCLOPROPYLMETHOXY-N-(3,5-DICHLORO-1-OXIDOPYRIDIN-4-YL)-5-(METHOXY)PYRIDINE-2-CARBOXAMIDE FOR THE TREATMENT OF MOTOR DISORDERS RELATED TO PARKINSON'S DISEASE

(75) Inventors: Philippe DELAY-GOYET, Paris (FR); Claire DELGORGE, Toulouse (FR); Christine MENET, Balma (FR); Gilles POUGHON, Saint Antonin Noble-Val (FR); Christine RAVINET-TRILLOU, Launaguet (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/573,326

(22) Filed: Oct. 5, 2009

Related U.S. Application Data
(63) Continuation of application No. PCT/FR2008/000534, filed on Apr. 16, 2008.

(30) Foreign Application Priority Data
Apr. 19, 2007 (FR) 0702853

(51) Int. Cl. A61K 31/444; A61P 25/16

(52) U.S. Cl. 514/335

(57) ABSTRACT

The present invention relates to the use of 4-cyclopropylmethoxy-N-(3,5-dichloro-1-oxidopyridin-4-yl)-5-(methoxy)pyridine-2-carboxamide in the form of a hydrate, of a solvate, of a base or of an addition salt with an acid, for preparing a medicament for use in the treatment of motor disorders related to Parkinson's disease.

The present application is a Continuation of International Application No. PCT/FR2008/000534, filed Apr. 16, 2008, which is incorporated herein by reference in its entirety.

The present invention relates to the use of 4-cyclopropylmethoxy-N-(3,5-dichloro-1-oxidopyridin-4-yl)-5-(methoxy)pyridine-2-carboxamide in the form of a hydrate, of a solvate, of a base or of an addition salt with an acid, for preparing a medicament for use in the treatment of motor disorders related to Parkinson's disease.

4-Cyclopropylmethoxy-N-(3,5-dichloro-1-oxidopyridin-4-yl)-5-(methoxy)pyridine-2-carboxamide, also called N-(3,5-dichloro-1-oxido-4-pyridinio)-4-cyclopropylmethoxy-5-methoxypyridine-2-carboxamide, is known to be part of the composition of medicaments for use in the treatment of various pathologies, including in particular inflammations of the joints, arthritis and rheumatoid arthritis, and for the treatment of memory disorders related to Alzheimer's disease. This compound, in hemihydrate form, is described, for example, in document WO 95/04045 (compound referenced FR).

There exists a need to find medicaments for treating patients preventively against the worsening of motor disorders which are related to Parkinson's disease. Studies in animals have found that a possible approach is the administration of compounds that can inhibit phosphodiesterases 4 (PDE 4), such as, for example, rolipram. However, clinical studies have shown that this compound, and also other PDE 4 inhibitors, induce emetic effects which prevent it from being used in therapy.

It has now been found that 4-cyclopropylmethoxy-N-(3,5-dichloro-1-oxidopyridin-4-yl)-5-(methoxy)pyridine-2-carboxamide can be used in the treatment of motor disorders related to Parkinson's disease, while at the same time avoiding the emetic effects at acceptable therapeutic doses.

A first subject of the invention therefore relates to the use of 4-cyclopropylmethoxy-N-(3,5-dichloro-1-oxidopyridin-4-yl)-5-(methoxy)pyridine-2-carboxamide for preparing a medicament for use in the treatment of motor disorders related to Parkinson's disease.

According to one embodiment of the invention, the use of 4-cyclopropylmethoxy-N-(3,5-dichloro-1-oxidopyridin-4-yl)-5-(methoxy)pyridine-2-carboxamide can be carried out in the form of a base or of an addition salt with an acid.

The salts that can be used in the context of the invention can be prepared with pharmaceutically acceptable acids, but the salts of other acids that are of use, for example, for the purification or the isolation of 4-cyclopropylmethoxy-N-(3,5-dichloro-1-oxidopyridin-4-yl)-5-(methoxy)pyridine-2-carboxamide are also part of the invention.

The use of 4-cyclopropylmethoxy-N-(3,5-dichloro-1-oxidopyridin-4-yl)-5-(methoxy)pyridine-2-carboxamide according to the invention can also be carried out in the form of a hydrate or of a solvate. The term “hydrate” or “solvate” is intended to mean the association or the combination of one or more molecules of 4-cyclopropylmethoxy-N-(3,5-dichloro-1-oxidopyridin-4-yl)-5-(methoxy)pyridine-2-carboxamide with one or more molecules of water or of solvent.

For the purpose of the present invention, the expression “motor disorder related to Parkinson's disease” is intended to mean the following disorders: bradykinesia, akinesia, rigidity, postural disorders and instability, impaired walking, tremors, problems with written and oral expression, dysphagia, respiratory problems, bladder and sphincter problems.

A second subject of the invention relates to a pharmaceutical composition comprising, as active ingredient, 4-cyclopropylmethoxy-N-(3,5-dichloro-1-oxidopyridin-4-yl)-5-(methoxy)pyridine-2-carboxamide, and one or more pharmaceutically acceptable excipients.

The composition used according to the invention comprises an effective dose of the active ingredient.

For example, the daily doses of active ingredient that can be used according to the invention are from 0.001 to 10 mg/day.

According to the usual practice, the dosage appropriate for each patient is determined by the physician according to the method of administration and the age, weight and response of said patient.

The doses depend on the desired effect, on the duration of the treatment and on the route of administration used.

There may be specific cases where higher or lower doses are appropriate. Such dosages do not depart from the context of the invention.

The excipients are selected, according to the pharmaceutical form and the method of administration desired, from the usual excipients which are known to those skilled in the art.

The composition may be administered orally, parenterally or rectally.

Appropriate unit administration forms comprise oral administration forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular, intravenous or intrathecal administration forms, rectal administration forms, and implants. For topical application, the active ingredients according to the invention can be used in creams, gels, ointments or lotions.

When a composition is prepared in tablet form, the active ingredient is mixed with one or more pharmaceutical excipients, such as gelatin, starch, lactose, magnesium stearate, talc, silica, gum Arabic, mannitol, microcrystalline cellulose, hypromellose, or the like.

The tablets may be coated with sucrose, with a cellulosic derivative or with other substances suitable for coating. The tablets may be prepared by various techniques, such as direct compression, dry or wet granulation or the hot-melt technique.

It is also possible to obtain a pharmaceutical composition in the form of a gel capsule by mixing the active ingredient with a diluent and transferring the mixture into soft or hard gel capsules.

For parenteral administration, use is made of aqueous suspensions, isotonic saline solutions or sterile and injectable solutions which contain pharmacologically compatible agents, for example propylene glycol or butylene glycol.

By way of example, a unit administration form of 4-cyclopropylmethoxy-N-(3,5-dichloro-1-oxidopyridin-4-yl)-5-(methoxy)pyridine-2-carboxamide in tablet form comprises the following ingredients:

| | |
|---|---|
| 4-Cyclopropylmethoxy-N- | 1 mg |
| (3,5-Dichloro-1-oxidopyridin-4-yl)- | |
| 5-(Methoxy)pyridine-2-carboxamide | |
| Mannitol | 224 mg |
| Sodium croscarmellose | 5 mg |
| Maize starch | 15 mg |
| Hydroxypropylmethylcellulose | 2 mg |
| Magnesium stearate | 3 mg |

The effects of the 4-cyclopropylmethoxy-N-(3,5-dichloro-1-oxidopyridin-4-yl)-5-(methoxy)pyridine-2-carboxamide used according to the invention were evaluated in mice using a model of dopaminergic neuron loss.

### EXAMPLE 1

### Protection in a Model of Parkinson's disease (Dopaminergic Neuron Loss in Mice Poisoned with MPTP (1-methyl-4-phenyl-1,2,3,6-tetrahydropyridine):

C57BL6 mice are given 4 intraperitoneal injections of 20 mg/kg of MPTP, each 2 hours apart. The 4-cyclopropylmethoxy-N-(3,5-dichloro-1-oxidopyridin-4-yl)-5-(methoxy)pyridine-2-carboxamide in solution in the carrier (methylcellulose (MC) (0.6%)+Tween-80 (0.5%)) is administered by gavage between the 2^{nd} and 3^{rd} injection of MPTP and just after the final injection of MPTP, and then twice a day for 17 days at the total daily doses of 0.015 and 0.050 mg/kg. Twenty-four hours after the final treatment, the striatum is removed and the dopamine uptake sites are quantified using a GBR12935 (1-[2-(diphenylmethoxy)ethyl]-4-(3-phenylpropyl)piperazine) binding method.

After injection of MPTP, the density of the dopamine uptake sites corresponds to only 58% (p<0.01) of that measured in the normal animals. The treatment with 4-cyclopropylmethoxy-N-(3,5-dichloro-1-oxidopyridin-4-yl)-5-(methoxy)pyridine-2-carboxamide showed an ability to protect against the decrease induced by the MPTP: the density of the dopamine uptake sites reaches 82% and 85% of the level observed in the normal animals, respectively, at the doses of 0.015 and 0.050 mg/kg/d (p<0.01 in comparison with the animals given only the MPTP).

### EXAMPLE 2

### Evaluation of the Emetic Effects of 4-cyclopropylmethoxy-N-(3,5-dichloro-1-oxidopyridin-4-yl)-5-(methoxy)pyridine-2-carboxamide.

The emetic capacity of 4-cyclopropylmethoxy-N-(3,5-dichloro-1-oxidopyridin-4-yl)-5-(methoxy)pyridine-2-carboxamide was evaluated in ferrets. Two groups of ferrets were used, the first being given the carrier (PEG200) and the second being given the 4-cyclopropylmethoxy-N-(3,5-dichloro-1-oxidopyridin-4-yl)-5-(methoxy)pyridine-2-carboxamide in solution in the carrier (PEG 200), by oral gavage. The animals were observed continually for 2 hours following administration, and then every hour until 6 hours after administration. The clinical signs (in particular, retching and vomiting) were noted.

When administered at 0.1 mg/kg, 4-cyclopropylmethoxy-N-(3,5-dichloro-1-oxidopyridin-4-yl)-5-(methoxy)pyridine-2-carboxamide induce neither retching nor vomiting in the 5 ferrets treated.

These results show that the administration of a therapeutic dose of 4-cyclopropylmethoxy-N-(3,5-dichloro-1-oxidopyridin-4-yl)-5-(methoxy)pyridine-2-carboxamide for treating motor disorders related to Parkinson's disease does not cause any emetic effect.

### EXAMPLE 3

### Evaluation of the Emetic Effects of (R)-(−)-rolipram (((4R)-4-[3-(cyclopentyloxy)-4-methoxyphenyl]pyrrolidine-2-one)).

The emetic capacity of (R)-(−)-rolipram was evaluated in the ferrets. Two groups of ferrets were used, the first being given the carrier (PEG200) and the second being given the 4-cyclopropylmethoxy-N-(3,5-dichloro-1-oxidopyridin-4-yl)-5-(methoxy)-pyridine-2-carboxamide in solution in the carrier (PEG 200), by oral gavage, at doses of 0.05 mg/kg and of 0.1 mg/kg. The animals were observed continually for the 2 hours following administration, and then once an hour up to 6 hours after the administration. The clinical signs were noted.

When administered at 0.05 mg/kg and 0.1 mg/kg, (R)-(−)-rolipram induce vomiting in the ferrets treated.

The results of example 3 show that the administration of a therapeutic dose of (R)-(−)-rolipram gives rise to emetic effects.

Thus, 4-cyclopropylmethoxy-N-(3,5-dichloro-1 -oxidopyridin-4-yl)-5-(methoxy)pyridine-2-carboxamide can be used in the preparation of a medicament for the treatment of motor disorders related to Parkinson's disease, while at the same time avoiding possible emetic effects.

We claim:

1. A method for treating a motor disorder related to Parkinson's disease, in a patient in need thereof, comprising administering to the patient a pharmaceutically effective amount of 4-cyclopropylmethoxy-N-(3,5-dichloro-1-oxidopyridin-4-yl)-5-(methoxy)pyridine-2-carboxamide, or a hydrate or solvate thereof, or an addition salt with an acid thereof.

2. A method for treating a motor disorder related to Parkinson's disease, in a patient in need thereof, comprising administering to the patient a pharmaceutically effective amount of 4-cyclopropylmethoxy-N-(3,5-dichloro-1-oxidopyridin-4-yl)-5-(methoxy)pyridine-2-carboxamide.
